(19) United States
(12) Patent Application Publication
(10) Pub. No.: US 2025/0049701 A1
(43) Pub. Date: Feb. 13, 2025

(54) SILK-BASED ARTICLES HAVING VARIABLE ACTIVE AGENT RELEASE RATES

(71) Applicant: Trustees of Tufts College, Medford, MA (US)

(72) Inventors: David L. Kaplan, Concord, MA (US); Junqi Wu, Medford, MA (US); Vincent P. Fitzpatrick, Somerville, MA (US)

(21) Appl. No.: 18/746,604

(22) Filed: Jun. 18, 2024

Related U.S. Application Data
(63) Continuation of application No. PCT/US22/82660, filed on Dec. 30, 2022.
(60) Provisional application No. 63/356,939, filed on Jun. 29, 2022.
(60) Provisional application No. 63/295,586, filed on Dec. 31, 2021.

(51) Int. Cl. A61K 9/00; A61K 47/34; A61K 47/42

(52) U.S. Cl. CPC A61K 9/0024; A61K 47/34; A61K 47/42

(57) ABSTRACT

Coated silk fibroin articles having variable active agent release rates and methods of making and using the same are disclosed. The coating can have variable water permeability. In some cases, the variable permeability is temperature-dependent. In these cases, the release rate of the active agent can be controlled by adjusting the water permeability by raising and lowering the temperature, so long as the temperature is maintained below a melting point of the coating. Elevating the temperature above the melting point causes loss of a portion of the coating, thereby permanently increasing the release rate.

### CROSS-REFERENCE TO RELATED APPLICATIONS

This application is a continuation of International Application Serial Number PCT/US2022/082660, filed Dec. 30, 2022, and published as WO 2023/130091. International Application Serial Number PCT/US2022/082660 is related to, claims priority to, and incorporates herein by reference for all purposes U.S. Provisional Patent Applications Nos. 63/295,586, filed Dec. 31, 2021, and 63/356,939, filed Jun. 29, 2022. Each of the foregoing patent applications is incorporated herein by reference in their entirety for all purposes.

### STATEMENT REGARDING FEDERALLY FUNDED RESEARCH

Not applicable.

### BACKGROUND

Control of release rates for local drug delivery remains an active area of research. New formulations and methods for tailoring release profiles are required.

Silk devices have been previously demonstrated as drug delivery platforms and functional medical implants, largely through the work of the inventors and collaborators. Previous efforts included encapsulating small molecules or larger/complex biological molecules in devices to achieve sustained release to target a variety of diseases, such as local chemotherapy drug treatment of tumors and local antibiotic treatment of appendicitis.

Traditionally, silk devices have struggled with providing sustained release, especially for hydrophilic molecules. Typically, silk devices rapidly absorb water when immersed in aqueous environments and rapidly release their drug molecules into the aqueous environment.

A need exists for new formulations that have controlled and/or sustained release properties and overcome the aforementioned shortcomings.

### SUMMARY

In an aspect, the present disclosure provides a method of delivering an active agent to a target location with a controlled release rate. The method includes: a) administering a coated silk fibroin article to a target location, the coated silk fibroin article having a solid silk fibroin dosage form core comprising an active agent and a polycaprolactone or other hydrophobic biocompatible polymer coating encapsulating the solid silk fibroin dosage form core; and b) subsequent to step a), elevating temperature of the coated silk fibroin article at the target location to above a first temperature threshold and below a second temperature threshold, wherein the second temperature threshold is a melting temperature of the polycaprolactone or other hydrophobic coating, wherein the first temperature threshold is a transition temperature below which the polycaprolactone coating is substantially water impermeable and above which the polycaprolactone coating is at least partly water permeable, thereby allowing water to pass between the target location and the solid silk fibroin dosage form core and providing a first active agent release rate of the active agent from the coated silk fibroin article to the target location.

In another aspect, the present disclosure provides a method of making a coated silk fibroin article. The method includes: a) encapsulating a solid silk fibroin dosage form core comprising an active agent with a polycaprolactone or other hydrophobic biocompatible polymer coating, the encapsulating optionally comprising dip-coating, 3D printing, spray coating, spin coating, flow coating and roll coating.

In yet another aspect, the present disclosure provides a coated silk fibroin article having controllable active agent release rates. The coated silk fibroin article includes a solid silk fibroin dosage form and a polycaprolactone or other hydrophobic and biocompatible polymer coating encapsulating the solid silk fibroin dosage form core. The solid silk fibroin dosage form core includes an active agent. A core-only comparison dosage form including the solid silk fibroin dosage form core including the active agent and lacking any coating has a core-only active agent release rate. The coated silk fibroin article has a first active agent release rate when the polymer coating is above a first temperature threshold and below a second temperature threshold. The second temperature threshold is a melting temperature of the polycaprolactone or other hydrophobic biocompatible polymer. The first temperature threshold is a transition temperature below which the polycaprolactone or other hydrophobic biocompatible polymer is substantially water impermeable and above which the polycaprolactone or other hydrophobic biocompatible coating is at least partly water permeable. The coated silk fibroin article has a second active agent release rate when the polycaprolactone or other hydrophobic biocompatible coating is below the first temperature threshold. The coated silk fibroin article loses at least part of the polycaprolactone or other hydrophobic biocompatible polymer coating when the polycaprolactone or other hydrophobic biocompatible coating is above the second temperature threshold and adopts a third active agent release rate between the first active agent release rate and the core-only active agent release rate. The third active agent release rate increases as more of the polycaprolactone or other hydrophobic biocompatible polymer coating is lost from the coated silk fibroin article.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTIONS OF THE DRAWINGS

FIG. 1 is a plot comparing release profiles with and without a polycaprolactone coating, as described in Example 1.

FIG. 2A is a graphic showing a method of making a coated dosage form, in accordance with aspect of the present disclosure.

FIG. 2B is a plot showing cumulative release at 37° C. and 50° C. for two identical compositions, as described in Example 2.

FIG. 3 is a plot showing release rates changing with temperature, as described in Example 2.

### DETAILED DESCRIPTION

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Approximately: as used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biocompatible: the term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

Biodegradable: as used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid) (PLA), poly(glycolic acid) (PGA), poly(lactic-co-glycolic acid) (PLGA), and copolymers with PEG, polyanhydrides, poly(ortho)esters, polyesters, polyurethanes, poly(butyric acid), poly(valeric acid), poly(caprolactone), poly(hydroxyalkanoates), poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Compaction: as used herein, the term “compaction” refers to a process by which a material progressively loses its porosity due to the effects of loading.

Composition: as used herein, may be used to refer to a discrete physical entity that comprises one or more specified components. In general, unless otherwise specified, a composition may be of any form—e.g., gas, gel, liquid, solid, etc. In some embodiments, “composition” may refer to a combination of two or more entities for use in a single embodiment or as part of the same article. It is not required in all embodiments that the combination of entities result in physical admixture, that is, combination as separate co-entities of each of the components of the composition is possible; however many practitioners in the field may find it advantageous to prepare a composition that is an admixture of two or more of the ingredients in a pharmaceutically acceptable carrier, diluent, or excipient, making it possible to administer the component ingredients of the combination at the same time.

Fusion: as used herein, the term “fusion” refers to a process of combining two or more distinct entities into a new whole.

Hydrophilic: as used herein, the term “hydrophilic” and/or “polar” refers to a tendency to mix with, or dissolve easily in, water.

Hydrophobic: as used herein, the term “hydrophobic” and/or “non-polar”, refers to a tendency to repel, not combine with, or an inability to dissolve easily in, water.

Improve, increase, or reduce: as used herein or grammatical equivalents thereof, indicate values that are relative to a baseline measurement, such as a measurement in a similar composition made according to previously known methods.

Macroparticle: as used herein, the term “macroparticle” refers to a particle having a diameter of at least 1 millimeter. In some embodiments, macroparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of macroparticles if the mean diameter of the population is equal to or greater than 1 millimeter.

Microparticle: as used herein, the term “microparticle” refers to a particle having a diameter between 1 micrometer and 1 millimeter. In some embodiments, microparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of microparticles if the mean diameter of the population is between 1 micrometer and 1 millimeter.

Nanoparticle: as used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer. In some embodiments, a population of particles is considered a population of nanoparticles if the mean diameter of the population is equal to or less than 1000 nm.

Physiological conditions: as used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Pure: as used herein, a material, additive, and/or entity is “pure” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure.

Reference: as used herein describes a standard or control relative to which a comparison is performed. For example, in some embodiments, a material, article, additive, entity or other sample, sequence or value of interest is compared with a reference or control material, article, additive, entity or other sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Solid form: as is known in the art, many chemical entities (in particular many organic molecules and/or many small molecules) can adopt a variety of different solid forms such as, for example, amorphous forms and/or crystalline forms (e.g., polymorphs, hydrates, solvates, etc.). In some embodiments, such entities may be utilized as a single such form (e.g., as a pure preparation of a single polymorph). In some embodiments, such entities may be utilized as a mixture of such forms.

Substantially: as used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### Compositions and Methods

The present disclosure provides coated silk fibroin articles and methods of making and using the same.

A method of delivering an active agent to a target location with a controlled release rate is provided. The method includes: a) administering a coated silk fibroin article to a target location, the coated silk fibroin article having a solid silk fibroin dosage form core comprising an active agent and a polycaprolactone or other hydrophobic biocompatible polymer coating encapsulating the solid silk fibroin dosage form core; and b) subsequent to step a), elevating temperature of the coated silk fibroin article at the target location to above a first temperature threshold and below a second temperature threshold, wherein the second temperature threshold is a melting temperature of the polycaprolactone or other hydrophobic biocompatible polymer coating, wherein the first temperature threshold is a transition temperature below which the polycaprolactone or other hydrophobic biocompatible polymer coating is substantially water impermeable and above which the polycaprolactone or other hydrophobic biocompatible polymer coating is at least partially water permeable, thereby allowing water to pass between the target location and the solid silk fibroin dosage form core and providing a first active agent release rate of the active agent from the coated silk fibroin article to the target location. The first temperature threshold can be higher than normal human body temperature. The first temperature threshold can be at least 20° C., at least 25° C., at least 30° C., at least 35° C., or at least 40° C.

The method can further include: c) subsequent to step b), reducing the temperature of the coated silk fibroin article at the target location to below the first temperature threshold, thereby returning the polycaprolactone or other hydrophobic biocompatible polymer coating to being substantially water impermeable and preventing water from passing between the target location and the solid silk fibroin dosage form core and providing a second active agent release rate of the active agent from the coated silk fibroin article to the target location. In other words, the switching between the first active agent release rate and the second active agent release rate is reversible simply by changing temperature.

The method can further include: c) subsequent to step b), elevating the temperature of the coated silk fibroin article at the target location to above the second temperature threshold, thereby at least partly melting the polycaprolactone coating and providing a third active agent release rate of the active agent from the coated silk fibroin article to the target location. The polycaprolactone or other hydrophobic biocompatible polymer coating can be removed by melting, which provides the maximum possible release rate. The second temperature threshold can be the melting point of pure polycaprolactone or other hydrophobic biocompatible polymer.

The method can include preventing the coated silk fibroin article from exceeding the second temperature threshold prior to step b).

A method of making a coated silk fibroin article is also disclosed. The method includes encapsulating a solid silk fibroin dosage form comprising an active agent with a polycaprolactone coating. The coating can be done by way of dip-coating, 3D printing, spray coating, spin coating, flow coating and roll coating. The coating can be tailored to provide desired thickness, which in turn can provide desired release rate properties. For example, multiple layers of dip-coating applications can be performed.

In some cases, the core can be a foam and the method can include forming the foam using methods known to those having ordinary skill in the art.

A method of making a coated silk fibroin article includes: (i) selecting an elevated temperature and/or an elevated pressure to produce a desired silk fibroin article core of a desired crystallinity and desired material properties; (ii) selecting a biodegradable polymer and a desired coating thickness to produce a desired coated silk fibroin article having at least one desired mechanical property or at least one desired water uptake, the selecting of step (ii) taking the desired crystallinity and the desired material properties into account; (iii) applying the elevated temperature and/or the elevated pressure to a silk fibroin material comprising substantially amorphous structure to form a silk fibroin article core, wherein the silk fibroin article core has the desired crystallinity and desired material properties; and (iv) coating the silk fibroin article core with a biodegradable hydrophobic polymer layer comprising the biodegradable polymer and having the desired coating thickness to form a coated silk fibroin article.

A method of making a coated silk fibroin article includes: (i) providing a silk fibroin material comprising substantially amorphous structure; (ii) applying at least one of elevated temperature and elevated pressure to the silk fibroin material to form a silk fibroin article core, wherein the applying induces fusion between at least a portion of the silk fibroin and structural change of fibroin in the silk fibroin material; and (iii) coating the silk fibroin article core with a biodegradable hydrophobic polymer layer to form a coated silk fibroin article.

A method of making a coated silk fibroin article includes: (i) selecting an elevated temperature and/or an elevated pressure to produce a desired silk fibroin article core of a desired crystallinity and desired material properties; (ii) applying the elevated temperature and/or the elevated pressure to a silk fibroin material comprising substantially amorphous structure to form a silk fibroin article core, wherein the silk fibroin article core has the desired crystallinity and the desired material properties; and (iii) coating the silk fibroin article core with a biodegradable hydrophobic polymer layer to form a coated silk fibroin article. The applying of step (ii) can induce fusion between at least a portion of the silk fibroin and structural change of fibroin in the silk fibroin material.

The coating step of any of the methods described herein can include three-dimensional printing, micro-scale mixing, spraying, dip coating, thermal molding (in concert with or in addition to the thermal molding that forms the core of the silk article), or a combination thereof. In reference to one specific, non-limiting example, relating to three-dimensional printing methods, the coating can be designed using software known to those having ordinary skill in the art (for example, CAD software, available from Solidworks, Waltham, MA), taking into account the dimensions of the selected silk articles. The thickness of the 3D-printed coating can be tuned using the software. In another specific, non-limiting example, relating to the case of micro-scale mixing, a powder of the coating material can be mixed with silk amorphous nanoparticles via analytical milling (20,000 rpm for 2 minutes using a Cole-Parmer (headquarters Vernon Hills, IL) analytical mill) and thermal molding. The different mixing ratio can affect the hydrophobicity of the final coated silk article. In yet another specific, non-limiting example, relating to the case of dip-coating, organic solvents in which silk is insoluble are used. The thickness of the coating is dependent on the concentration of the coating material, the number of layers, and the thickness of individual layers. The organic solvent can be removed through drying in a fume hood after the coating process. In the case of thermal spray coating, the thickness of the coating can depend on coating speed and spray layer thickness. In some cases, thermal molding itself can be used to form the coatings around preformed silk article cores. As an alternative, thermal molding could be used to form shells of the coating material, followed by filling the shells with silk and thermomolding to form the coated article.

In some embodiments, methods disclosed herein involve the fabrication of amorphous silk nanomaterials (ASN) generated from aqueous silk fibroin solution. ASN may then be treated by hot pressing, leading to fusion and densification of the silk (e.g., into a silk article core). The resulting silk bulk material exhibits specific strength higher than that of most natural structural materials and has been shown effective for fabricating silk-based composites. In addition, it is shown that the engineered silk material has thermoforming properties, which allows the materials to be further transformed to desirable shapes under proper conditions. In some embodiments, compositions and methods described herein demonstrate a thermal and pressure-based, time-efficient and controllable method to transform silk fibroin from a silk fibroin material including substantial amounts of amorphous silk fibroin (for example, in powder form) directly to bulk structural material, which can function as the cores described herein. In some embodiments, methods and compositions described herein may allow for the application of more traditional process and molding techniques to silk materials, where this was not previously successfully employed for silk. Additionally, in some embodiments, processing methods described herein avoid the need for solvent or aqueous approaches, and providing direct routes to transform silk fibroin material into part cores. In accordance with various embodiments, methods described herein provide for the transformation of silk fibroin from amorphous materials to a semi-crystalline high-performance structural material through controlled application of heat and pressure. In some embodiments, provided processes induce a conformation transition of silk molecules from random coil to β-sheet. In some embodiments, provided methods include the processing of natural silk fiber into amorphous silk material (e.g., powder) via degumming, silk fibroin solubilization and freeze drying to prepare the proper premolding materials; feeding the amorphous silk material into a predesigned mold; and inducing the conformation and structure change of silk by applying heat and pressure. Additionally, this method can be processed with silk alone, or with the addition of inorganic fillers or second polymers to generate composite devices.

In some cases, the methods described herein can include selecting an elevated temperature and an elevated pressure to produce a desired silk fibroin article core of a desired crystallinity and desired material properties and then applying that elevated temperature and elevated pressure to a silk fibroin material having substantially amorphous structure. That is, the methods described herein can predictably select and apply temperatures and pressures to produce article cores having desired crystallinity and material properties.

### Coated Silk Fibroin Articles

In some cases, the coated silk fibroin articles described herein have controllable active release rates. In these cases, the coated silk fibroin article can include a solid silk fibroin dosage form core comprising an active agent and a polycaprolactone or other hydrophobic biocompatible polymer coating encapsulating the solid silk fibroin dosage form core.

The solid silk fibroin dosage form core comprising the active agent has a core-only active agent release rate. This serves as a “baseline” release rate and is the fastest release rate achievable from the articles disclosed herein. In other words, the complete removal of the polycaprolactone coating provides the highest release rate.

The coated silk fibroin article can have a first active agent release rate when the polycaprolactone coating is above a first temperature threshold and below a second temperature threshold. In this temperature window, the polycaprolactone or other hydrophobic biocompatible polymer coating is above a crystalline transition temperature, which allow some degree of water permeability, but the polycaprolactone coating does not melt and retains physical integrity.

The coated silk fibroin article can have a second active agent release rate when the polycaprolactone or other hydrophobic biocompatible polymer coating is below the first temperature threshold. In some cases, the second active agent release rate is effectively zero.

Raising the coated silk fibroin article above the second temperature threshold causes melting of the polycaprolactone or other hydrophobic biocompatible polymer coating and removal of at least a portion of the coating. This increases the active agent release rate to a release rate between the first active agent release rate and the core-only active agent release rate.

When the article is coated with a polycaprolactone or other hydrophobic biocompatible polymer coating, the polycaprolactone or other hydrophobic biocompatible polymer can have a number average molecular weight of at least 5 kDa, at least 10 kDa, at least 20 kDa, at least 40 kDa, at least 50 kDa, at least 60 kDa, or at least 70 kDa. The polycaprolactone can have a number average molecular weight of at most 500 kDa, at most 250 kDa, or at most 150 kDa.

The coated silk fibroin articles described herein can include a silk fibroin article core and a biodegradable hydrophobic polymer layer substantially encompassing the silk fibroin article core. The silk fibroin article core can be a thermally-processed silk fibroin article core or a pressure-processed silk fibroin article core, as described herein.

The present disclosure includes multiple related technical advances with respect to the production of silk fibroin materials that have improved mechanical properties. The general idea of coating silk fibroin materials has been previously contemplated, including efforts to enhance hydrophobicity with methods including one-step atomic layer deposition of TiO₂, plasma-polymerized hexamethyldisiloxane (HMDSO) deposition, and surface modification with nano-SiO₂. The inventors discovered that a biodegradable hydrophobic polymer coating (i.e., a specific class and quality of coating) that is applied to a thermally-processed silk fibroin articles (i.e., a specific type of article that is made from a specific material) provides unexpectedly large improvements in multiple performance characteristics that are relevant to the use of thermally-processed silk fibroin articles as mechanically-strong biomaterials. Two prominent improvements are an impressive reduction in the water uptake of articles and the mechanical properties as measured by a three-point bending test. The reduction in water uptake is unexpectedly improved relative to other approaches that the inventors tried before the inventive combination disclosed herein. The improved mechanical properties are unexpectedly large in scale. The uncoated thermally-processed silk fibroin article has mechanical properties that are inferior to polylactide, a conventional biodegradable hydrophobic polymer that has shortcomings that are described elsewhere herein, having similar size and shape. Specifically, the flexural strain (as measured in a three-point bending test) that can be achieved by polylactide, and other conventional biodegradable hydrophobic polymers, is orders of magnitude greater than can be achieved by an uncoated thermally-processed silk article. However, the coated silk fibroin article disclosed herein has mechanical properties that are compatible to polylactide.

Thermally- and pressure-processed silk articles can have limited utility in applications that have strict mechanical property requirements, due at least in part to the uptake of water, which can compromise the mechanical properties in aqueous environments. Uncoated silk fibroin bone screws made by the methods described herein for making silk fibroin article cores have been reported to absorb up to 35% by weight of water when immersed in a PBS solution at 37° C. for 8 hours. This water uptake results in swelling of the articles and reduction in some of the mechanical properties, including stiffness. The disclosed articles and methods overcome some of these shortcomings.

The biodegradable hydrophobic polymer layer can comprise aliphatic polymers, such as polylactide, polycaprolactone, and polycarbonate, aromatic and aliphatic polyanhydrides, polyurethanes, polyamides, poly(ester amide), or a copolymer or combination thereof. In some cases, the biodegradable hydrophobic polymer layer includes polylactide or polycaprolactone.

In some cases, the biodegradable hydrophobic polymer layer can have a substantially uniform thickness. The average thickness of the biodegradable hydrophobic polymer layer can be between 1 μm and 5 mm.

The biodegradable hydrophobic polymer layer can account for no more than 50%, no more than 25%, no more than 15%, or no more than 10% by weight of the coated silk fibroin article. The biodegradable hydrophobic polymer layer can account for at least 1% by weight of the coated silk fibroin article. The specific weight of the biodegradable hydrophobic polymer layer can be dependent on the chosen coating method or the desired mechanical properties.

The biodegradable hydrophobic polymer layer can comprise an acid-activated protease. The acid-activated protease can be selected from the group consisting of pepsin, a cathepsin, renin, and combinations thereof. The acid-activated protease is triggered by a local pH reduction initiated by dissolution or degradation of the biodegradable hydrophobic polymer layer. It should be appreciated that this local pH reduction is typically considered a shortcoming of the materials included in the biodegradable hydrophobic polymer layer, because of the biological inflammation response that it triggers. But in this case, the local pH reduction is used as a trigger to initiate the degradation of the material itself. The acid-activated protease can be present in the biodegradable hydrophobic polymer layer in an amount by weight of between 0.1 wt % and 5.0 wt %, including but not limited to, between 0.5 wt % and 4.0 wt %, or between 1.0 wt % and 3.0 wt %.

### Coated Articles

The present disclosure also provides coated article where the silk fibroin core is replaced with a different core that is composed of different materials. For example, the solid silk fibroin dosage form core described herein can be another, different sustained release core formulation, as will be appreciated by those having ordinary skill in the art. All other aspects of the disclosure are applicable.

### Silk Materials

Any of a variety of silk materials may be used in accordance with various embodiments. In some embodiments, a silk material may be or comprise silk fibroin (e.g., degummed or substantially sericin free silk fibroin). In some embodiments, a silk material may be or comprise silk powder (e.g., comprising a plurality of silk particles).

In some embodiments, a silk fibroin material may be or comprise silk particles (e.g., microparticles or nanoparticles). As used herein, the term “particles” includes spheres, rods, shells, prisms, and related structures. While any application-appropriate particle size is contemplated as within the scope of the present disclosure, in some embodiments, a silk particle be have a diameter between 1 nm and 1,000 μm (e.g., between 1 nm and 1 μm, between 1 μm and 1,000 μm, etc.). In some embodiments, a silk particle may have a diameter of greater than 1,000 μm.

Various methods of producing silk particles (e.g., nanoparticles and microparticles) are known in the art. For example, a milling machine (e.g., a Retsch planetary ball mill) can be used to produce silk powder. Generally, the ball mill consists of either two or four sample cups arranged around a central axis, which is geared such that each cup rotates both centrally and locally. Each ceramic cup is filled with small ceramic spheres. A range of sizes is available; balls with a diameter of 10 millimeters were/are used for the milling operations described in the present disclosure. As the cups spin, the spheres crush material in the cups to a small characteristic size. Both degummed and non-degummed silk can be converted from pulverized material to powder form in the ball mill.

In other embodiments, alternative powder formation techniques can be used (e.g., lyophilization or flash freezing and crushing). In other embodiments, alternative grates on the pulverizer, with larger holes, can be used. This can generate larger silk particle sizes.

In some embodiments, silk particles can be produced using a freeze-drying method as described in U.S. Provisional Application Ser. No. 61/719,146, filed Oct. 26, 2012, content of which is incorporated herein by reference in its entirety. Specifically, silk foam can be produced by freeze-drying a silk solution. The foam then can be reduced to particles. For example, a silk solution can be cooled to a temperature at which the liquid carrier transforms into a plurality of solid crystals or particles and removing at least some of the plurality of solid crystals or particles to leave a porous silk material (e.g., silk foam). After cooling, liquid carrier can be removed, at least partially, by sublimation, evaporation, and/or lyophilization. In some embodiments, the liquid carrier can be removed under reduced pressure. After formation, the silk fibroin foam can be subjected to grinding, cutting, crushing, or any combinations thereof to form silk particles. For example, the silk fibroin foam can be blended in a conventional blender or milled in a ball mill to form silk particles of desired size.

In some embodiments, the silk fibroin material comprising substantial amounts of amorphous structure is prepared from silk solution and is composed of nanostructures, an may be referred to as nano-sized silk powder (NSP) and be part of materials referred to amorphous silk nanomaterials (ASN). As used herein, these terms are equivalent and may be used interchangeably.

Without wishing to be held to a particular theory, in some embodiments, the present disclosure encompasses the recognition that the use of particular starting materials (e.g., silk fibroin material comprising substantial amounts of amorphous structure) allows for the production of previously unattainable compositions. In some embodiments, a silk material is not made from solubilized silk. In some embodiments, a silk material may be lyophilized.

### Silk Fibroin

According to various embodiments, any silk fibroin may be used in provided methods. In some embodiments, the silk fibroin is selected from the group consisting of spider silk (e.g., from Nephila clavipes), silkworm silk (e.g., from Bombyx mori), and recombinant silks (e.g., produced/engineered from bacterial cells, yeast cells, mammalian cells, transgenic animals, and/or transgenic plants). In accordance with various embodiments, silk used in provided methods and compositions is degummed silk (i.e. silk fibroin with at least a portion of the native sericin removed). Degummed silk can be prepared by any conventional method known to one skilled in the art. For example, B. mori cocoons are boiled for a period of pre-determined time in an aqueous solution. Generally, longer degumming time generates lower molecular silk fibroin. In some embodiments, the silk cocoons are boiled for at least 60 minutes, at least 70 minutes, at least 80 minutes, at least 90 minutes, at least 100 minutes, at least 110 minutes, at least 120 minutes, or longer. Additionally or alternatively, in some embodiments, silk cocoons can be heated or boiled at an elevated temperature. For example, in some embodiments, silk cocoons can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher. In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, the aqueous solution used in the process of degumming silk cocoons comprises about 0.02M Na₂CO₃. The cocoons are rinsed, for example, with water to extract the sericin proteins. The degummed silk can be dried and used for preparing silk powder. Alternatively, the extracted silk can be dissolved in an aqueous salt solution. Salts useful for this purpose include lithium bromide, lithium thiocyanate, calcium nitrate or other chemicals capable of solubilizing silk. In some embodiments, the extracted silk can be dissolved in about 8M-12 M LiBr solution. The salt is consequently removed using, for example, dialysis.

In some embodiments, the silk fibroin is substantially depleted of its native sericin content (e.g., 5% (w/w) or less residual sericin in the final extracted silk). In some embodiments, the silk fibroin is entirely free of its native sericin content. As used herein, the term “entirely free” (i.e. “consisting of” terminology) means that within the detection range of the instrument or process being used, the substance cannot be detected or its presence cannot be confirmed. In some embodiments, the silk fibroin is essentially free of its native sericin content. As used herein, the term “essentially free” (or “consisting essentially of”) means that only trace amounts of the substance can be detected, is present in an amount that is below detection, or is absent.

If necessary, a silk solution may be concentrated using, for example, dialysis against a hygroscopic polymer, for example, PEG, a polyethylene oxide, amylose or sericin. In some embodiments, the PEG is of a molecular weight of 8,000-10,000 g/mol and has a concentration of about 10% to about 50% (w/v). A slide-a-lyzer dialysis cassette (Pierce, MW CO 3500) can be used. However, any dialysis system can be used. The dialysis can be performed for a time period sufficient to result in a final concentration of aqueous silk solution between about 10% to about 30%. In most cases dialysis for 2-12 hours can be sufficient. See, for example, International Patent Application Publication No. WO 2005/012606, the content of which is incorporated herein by reference in its entirety. Another method to generate a concentrated silk solution comprises drying a dilute silk solution (e.g., through evaporation or lyophilization). The dilute solution can be dried partially to reduce the volume thereby increasing the silk concentration. The dilute solution can be dried completely and then dissolving the dried silk fibroin in a smaller volume of solvent compared to that of the dilute silk solution. In some embodiments, a silk fibroin solution can optionally, at a suitable point, be filtered and/or centrifuged. For example, in some embodiments, a silk fibroin solution can optionally be filtered and/or centrifuged following the heating or boiling step. In some embodiments, a silk fibroin solution can optionally be filtered and/or centrifuged following the dialysis step. In some embodiments, a silk fibroin solution can optionally be filtered and/or centrifuged following the step of adjusting concentrations. In some embodiments, a silk fibroin solution can optionally be filtered and/or centrifuged following the step of reconstitution. In any of such embodiments, the filtration and/or centrifugation step(s) can be carried out to remove insoluble materials. In any of such embodiments, the filtration and/or centrifugation step(s) can be carried out to selectively enrich silk fibroin fragments of certain molecular weight(s).

In some embodiments, silk fibroin and/or a silk fibroin article core, may comprise a protein structure that substantially includes β-turn and/or β-strand regions. Without wishing to be bound by a theory, the silk β sheet content can impact gel function and in vivo longevity of the composition. It is to be understood that composition including non-β sheet content (e.g., e-gels) can also be utilized. In some embodiments, silk fibroin has a protein structure including, e.g., about 5% β-turn and β-strand regions, about 10% β-turn and β-strand regions, about 20% β-turn and β-strand regions, about 30% β-turn and β-strand regions, about 40% β-turn and β-strand regions, about 50% β-turn and β-strand regions, about 60% β-turn and β-strand regions, about 70% β-turn and β-strand regions, about 80% β-turn and β-strand regions, about 90% β-turn and β-strand regions, or about 100% β-turn and β-strand regions. In other aspects of these embodiments, silk fibroin has a protein structure including, e.g., at least 10% β-turn and β-strand regions, at least 20% β-turn and β-strand regions, at least 30% β-turn and β-strand regions, at least 40% β-turn and β-strand regions, at least 50% β-turn and β-strand regions, at least 60% β-turn and β-strand regions, at least 70% β-turn and β-strand regions, at least 80% β-turn and β-strand regions, at least 90% β-turn and β-strand regions, or at least 95% β-turn and β-strand regions. In yet other aspects of these embodiments, silk fibroin has a protein structure including, e.g., about 10% to about 30% β-turn and β-strand regions, about 20% to about 40% β-turn and β-strand regions, about 30% to about 50% β-turn and β-strand regions, about 40% to about 60% β-turn and β-strand regions, about 50% to about 70% β-turn and β-strand regions, about 60% to about 80% β-turn and β-strand regions, about 70% to about 90% β-turn and β-strand regions, about 80% to about 100% β-turn and β-strand regions, about 10% to about 40% β-turn and β-strand regions, about 30% to about 60% β-turn and β-strand regions, about 50% to about 80% β-turn and β-strand regions, about 70% to about 100% β-turn and β-strand regions, about 40% to about 80% β-turn and β-strand regions, about 50% to about 90% β-turn and β-strand regions, about 60% to about 100% β-turn and β-strand regions, or about 50% to about 100% β-turn and β-strand regions. In some embodiments, silk β sheet content, from less than 10% to ˜55% can be used in the silk fibroin compositions disclosed herein.

In some embodiments, silk fibroin, or a silk fibroin article core, has a protein structure that is substantially-free of α-helix and/or random coil regions. In aspects of these embodiments, the silk fibroin has a protein structure including, e.g., about 5% α-helix and/or random coil regions, about 10% α-helix and/or random coil regions, about 15% α-helix and/or random coil regions, about 20% α-helix and/or random coil regions, about 25% α-helix and/or random coil regions, about 30% α-helix and/or random coil regions, about 35% α-helix and/or random coil regions, about 40% α-helix and/or random coil regions, about 45% α-helix and/or random coil regions, or about 50% α-helix and/or random coil regions. In other aspects of these embodiments, the silk fibroin has a protein structure including, e.g., at most 5% α-helix and/or random coil regions, at most 10% α-helix and/or random coil regions, at most 15% α-helix and/or random coil regions, at most 20% α-helix and/or random coil regions, at most 25% α-helix and/or random coil regions, at most 30% α-helix and/or random coil regions, at most 35% α-helix and/or random coil regions, at most 40% α-helix and/or random coil regions, at most 45% α-helix and/or random coil regions, or at most 50% α-helix and/or random coil regions. In yet other aspects of these embodiments, the silk fibroin has a protein structure including, e.g., about 5% to about 10% α-helix and/or random coil regions, about 5% to about 15% α-helix and/or random coil regions, about 5% to about 20% α-helix and/or random coil regions, about 5% to about 25% α-helix and/or random coil regions, about 5% to about 30% α-helix and/or random coil regions, about 5% to about 40% α-helix and/or random coil regions, about 5% to about 50% α-helix and/or random coil regions, about 10% to about 20% α-helix and/or random coil regions, about 10% to about 30% α-helix and/or random coil regions, about 15% to about 25% α-helix and/or random coil regions, about 15% to about 30% α-helix and/or random coil regions, or about 15% to about 35% α-helix and/or random coil regions.

### Elevated Temperatures

As discussed herein, provided methods and compositions include the exposure to elevated temperature(s). As used herein, the term “elevated temperatures” refers to temperatures higher than standard room temperature (i.e., greater than 25° C.). In some embodiments, provided methods or compositions include exposure to a single elevated temperature. In some embodiments, provided methods or compositions include exposure to at least two elevated temperatures (e.g., 3, 4, 5, 6, 7, 8, 9, 10 or more). In some embodiments where a method of composition includes two or more elevated temperatures, at least two of those elevated temperatures are different from one another.

In some embodiments, an elevated temperature may be between 25° C. and 200° C. By way of specific exemplary ranges, in some embodiments, an elevated temperature may be between 25° C. and 150° C., between 25° C. and 100° C., between 25° C. and 95° C., between 25° C. and 50° C., between 50° C. and 200° C., between 50° C. and 150° C., between 50° C. and 100° C., between 25° C. and 100° C. between 125° C. and 200° C., or any other range between 125° C. and 175° C.

In some embodiments, an elevated temperature may be at least 25° C. By way of additional example, in some embodiments, an elevated temperature may be at least 26° C., 27° C., 28° C., 29° C., 30° C., 35° C., 40° C., 45° C., 50° C., 55° C., 60° C., 65° C., 70° C., 75° C., 80° C., 85° C., 90° C., 95° C. or 100° C. In some embodiments, enhanced crystallization of silk fibroin material is observed at temperatures at or above 95° C.

In some embodiments, an elevated temperature may be at most 125° C. By way of additional example, in some embodiments, an elevated temperature may be at most 126° C., 127° C., 128° C., 129° C., 130° C., 135° C., 140° C., 145° C., 150° C., 155° C., 160° C., 165° C., 170° C., 175° C., 180° C., 185° C., 190° C., or 195° C.

Application of elevated temperature(s) to a provided composition or in a provided method may occur in any application-appropriate manner. By way of non-limiting example, in some embodiments, application of elevated temperature(s) may be via heat pressing, via a heating device such as an oven, heating stage, exposed flame or other mechanism.

Application of elevated temperature(s) may occur at or over any of a variety of time periods. For example, in some embodiments, application of elevated temperature(s) occurs substantially instantly (e.g., by placement over a flame or in an oven). In some embodiments, application of elevated temperature(s) occurs over a period of seconds, minutes, or hours. In some embodiments, application of elevated temperature(s) occurs over a period of time between 1 second and 1 hour.

### Elevated Pressure

As discussed herein, provided methods and compositions include the exposure to elevated pressure(s). As used herein, the term “elevated pressures” refers to pressures higher than standard atmospheric pressure (i.e., 1.013 bar). In some embodiments, provided methods or compositions include exposure to a single elevated pressure. In some embodiments, provided methods or compositions include exposure to at least two elevated pressures (e.g., 3, 4, 5, 6, 7, 8, 9, 10 or more). In some embodiments where a method of composition includes two or more elevated pressures, at least two of those elevated pressures are different from one another.

Any application-appropriate method(s) may be used to cause elevated pressure as applied to provided compositions or in provided methods. By way of non-limiting example, in some embodiments, elevated pressure may include use of a vacuum, a press (e.g. heat press), and combinations thereof.

In some embodiments, application of elevated pressure may be or include uniaxial compression. In some embodiments, application of elevated pressure may be or include multi-axial compression (e.g., biaxial compression).

While any application-appropriate level of elevated pressure may be used, in some embodiments, an elevated pressure between 1 MPa and 1 GPa is used. By way of specific exemplary ranges, in some embodiments, an elevated pressure may be between 10 MPa and 1 GPa, between 50 MPa and 1 GPa, between 100 MPa and 1 GPa, between 200 MPa and 1 GPa, between 300 MPa and 1GP, between 400 MPa and 1 GPa or between 500 MPa and 1 GPa. In some embodiments, an elevated pressure may be or comprise at least 1 MPa (e.g., at least 2 MPa, 3 MPa, 4 MPa, 5 MPa, 6 MPa, 7 MPa, 8 MPa, 9 MPa, 10 MPa, 20 MPa, 30 MPa, 40 MPa, 50 MPa, 60 MPa, 70 MPa, 80 MPa, 90 MPa, 100 MPa 150 MPa, 200 MPa, 250 MPa, 300 MPa, 350 MPa, 400 MPa, 450 MPa, 500 MPa, 550 MPa, 600 MPa, 650 MPa, 700 MPa, or 750 MPa).

### Silk Article Cores

In some embodiments, provided silk article cores exhibit a substantially homogenous structure. As used herein, “substantially homogenous structure” means that silk fibroin molecules are distributed and/or configured in a consistent way throughout substantially all of a portion of or the entirety of an article. Further, in some embodiments, silk article cores may exhibit significant amounts of silk fibroin in a semi-crystalline structure. In some embodiments, production of a silk article core according to provided methods includes a transition on the structure of silk fibroin from a substantially amorphous state to a semi-crystalline state, for example, as observed via X-ray diffraction.

In some embodiments, a silk article core may exhibit significant amounts of 3-sheet structure. For example, in some embodiments, a silk article core may exhibit at least 10 wt % more (e.g., at least 20 wt %, 30 wt %, 40 wt %) 3-sheet structures as compared to the starting silk fibroin material. In some embodiments, a silk article core may exhibit at least 50 wt % more (e.g., at least 60 wt %, 70 wt %, 80 wt %, 90 wt %, 95 wt %) 3-sheet structures as compared to the starting silk fibroin material.

In some embodiments, crystallinity of silk article cores may be controlled by the application of temperature and pressure. For example, in some embodiments, when amorphous silk is processed at temperatures ranging from about 25° C.-125° C., the silk article core may contain about 10-15% j-sheet structures. In some embodiments when amorphous silk is processed at temperatures ranging from about 125° C.-175° C., the silk article core may contain for example, about 20-35% 3-sheet structures or for example, over 40% 3-sheet structures.

In some embodiments, provided methods and compositions allow for the production of silk article cores which that are homogenous, where the silk amorphous powders are packed together via the bonding between neighboring raw silk powders, for example, at processing temperatures of about 25° C.-95° C. In some embodiments, provided methods and compositions allow for the production of silk article cores which that are homogenous, where the silk molecules of amorphous powders gain more mobility as they are heated above the glass transition temperature and self-assemble into interlocked nanoglobules, for example, at processing temperatures of about 125° C.-175° C.

In some embodiments, provided methods and compositions allow for the production of silk article cores (e.g., thin films) that undergo thermal softening and are bendable and moldable into a desired shape. In some embodiments, provided methods and compositions allow for the production of silk article cores that are machinable.

Provided methods and compositions allow for the production of complex silk article cores in ways that were not achievable using previous methods (e.g., silk screws that can resist torsion forces relevant to in vivo use). By way of non-limiting example, in some embodiments provided methods and compositions may be used to produce silk article cores such as films, fibers, meshes, needles, tubes, plates, screws, rods, and any combination thereof.

In some embodiments, a silk article core may be amenable to one or more types of patterning. In some embodiments, patterning may be or comprise macropatterning. In some embodiments, patterning may be or comprise micropatterning (i.e., patterning with micro scale features). In some embodiments, patterning may be or comprise nanopatterning (i.e., patterning with nano scale features). In some embodiments, patterning may be or comprise: etching, lithography-based patterning, carving, cutting, and any combination thereof.

In some embodiments, a silk article core may be subjected to one or more types of processing (e.g., 2, 3, 4, 5, 6, 7, 8, 9, 10 or more). While any application-appropriate form of processing is contemplated as within the scope of the present disclosure, in some embodiments, processing may be or comprise machining, rolling, drilling, milling, sanding, punching die cutting, extruding, chemical etching, coating, molding, turning, thread rolling, and any combination thereof.

### Exemplary Properties or Characteristics of Silk Article Cores

In some embodiments, provided compositions (e.g., silk article cores) may be substantially transparent. In some embodiments, provided compositions (e.g., silk article cores) may be semi-transparent. In some embodiments, provided compositions (e.g., silk article cores) may be substantially non-transparent. As used herein, the term “transparent” refers to the propensity of an object to transmit light (with or without scattering of said light). In some embodiments, a composition/article is said to be substantially transparent if it transmits ≥80% of light it is exposed to in the visible range (400 nm-800 nm). In some embodiments, a composition/article is said to be semi-transparent if it transmits between 50%-80% of light it is exposed to in the visible range (400 nm-800 nm). In some embodiments, a composition/article is said to be substantially non-transparent if it transmits ≤50% of light it is exposed to in the visible range (400 nm-800 nm).

In some embodiments, provided compositions may be biocompatible and/or biodegradable. In some embodiments, provided compositions may exhibit particular degradation profile(s). By way of specific example, in some embodiments, a provided composition may degrade at least 50 wt % after about 96 hours of exposure to an aqueous environment at 37° C. In some embodiments, a provided composition may not degrade more than 10% after months of exposure to an in vivo environment or condition.

In some embodiments, provided compositions may exhibit one or more desirable properties including, but not limited to: electrical conductivity, enhanced machinability, and/or enhanced thermoformability.

### Active Agents

The silk fibroin cores described herein can include one or more active agents. The active agents can be selected from those understood by a skilled artisan to have therapeutic benefit.

As used herein, the term “active agent” refers to any molecule, compound or composition, bioactivity of which is desired to be stabilized when such molecule, compound, or composition is subjected to at least one state-changing cycle, and/or is maintained under certain conditions as described herein. For the methods and compositions described herein, any active agent that can be maintained within a silk-fibroin matrix and which can diffuse through the disclosed coating are suitable for use. Examples of active agents include, but are not limited to, proteins, peptides, antigens, immunogens, vaccines, antibodies or portions thereof (e.g., antibody-like molecules), enzymes, nucleic acids (e.g., oligonucleotides, polynucleotides, siRNA, shRNA), aptamers, viruses, bacteria, small molecules, cells, photo synthetic and energy-harvesting compounds, flavors, antibiotics, therapeutic agents, diagnostic agents such as contrast agents or dye, viral vectors, and anti-venom.

In some cases, the active agent has a molecular weight that is below a diffusion threshold, such that the active agent can suitably diffuse through the polycaprolactone or other hydrophobic biocompatible polymer coating when the coating is at least partly water permeable.

In some cases, the active agent can have a molecular weight of 10 kg/mol or less, 5 kg/mol or less, 1 kg/mol or less, or 750 g/mol or less.

The release rates of active agent described herein are generally associated with aqueous environments, but are not intended to be limited to a specific environment. For example, the release rate can be a release rate into pure water or into simulated gastric fluid. The release rates can be measured at a variety of temperatures that fall within the broad categories disclosed in ways understood to those having ordinary skill in the art. In general, the release rates described herein are described relative to one another rather than in absolute terms, because the concepts are applicable to all but the most rapid active agent release profiles and can be deployed in a variety of physical contexts without departing from the spirit of the invention. For the avoidance of doubt, the present disclosure is intended to cover compositions and methods that involve the disclosed relative release rates, regardless of the absolute release rates. If absolute release rates are provided, they are intended to provide context to the relative rates and are not intended to be limiting.

### Additives

In some embodiments, provided methods and compositions include one or more additives (e.g., 2, 3, 4, 5, 6, 7, 8, 9, 10 or more). In some embodiments, at least one additive may be mixed with or otherwise associated with a silk fibroin material prior to an applying step (e.g. exposure to one or more of elevated temperature and elevated pressure). In some embodiments, at least one additive may be mixed with or otherwise associated with a silk fibroin material substantially at the same time as an applying step). In some embodiments, at least one additive may be mixed with or otherwise associated with a silk fibroin material subsequently to an applying step.

Provided methods and compositions are amenable to the addition of any of a variety of additives. By way of non-limiting example, in some embodiments an additive may be or comprise a small molecule, an organic macromolecule, an inorganic macromolecule, an electrically conductive material, an inorganic material, a hydrophobic material, a hydrophilic material, a nanomaterial, and any combination thereof.

The processing of the silk-based materials, including pure silk materials and silk-based composite materials, can be modified with addition of one or more additives. In some embodiments, a function of an additive may be to tune the processing conditions and the properties of the products. In some embodiments, additives may be selected from water; glycerol; saccharides; biological macromolecules, e.g. peptide, proteins; antibodies and antigen binding fragments; nucleic acids; immunogens; antigens; enzyme; synthetic polymers, e. g. poly(ethylene) glycol, poly-lactic acid, poly(lactic-co-glycolic acid) to name but a few specific examples, though any application-appropriate additive is specifically contemplated as within the scope of the present disclosure.

In some embodiments, for example some embodiments contemplated for in vivo use, provided compositions may comprise one or more proteases. In some embodiments, an organic macromolecule is or comprises at least one protease. In some embodiments, a protease is or comprises one or more of Proteinase XIV, Proteinase K, α-chymotrypsin, collagenase, matrix metalloproteinase-1 (MMP-1), and MMP-2. In some embodiments, a protease may be useful in tailoring the degradation profile of a particular provided composition (e.g., in an in vivo environment).

In some embodiments, an electrically conductive material may be or comprise an organic conductive material and/or an inorganic conductive material (e.g., a metal). In some embodiments, an electrically conductive material may be or comprise at least one of a conductive polymer, graphene, silver, gold, aluminum, copper, platinum, steel, brass, bronze, and iron oxide.

Any application-appropriate amount of one or more additives may be useful according to various embodiments. By way of non-limiting example, in some embodiments, an additive may be present in a provided composition in an amount between 0.001 wt % and 95 wt %. In some embodiments, one or more additives may be mixed with a silk fibroin material in an amount ranging between 0.001 wt % and 95 wt % of the silk fibroin material.

### EXAMPLES

### Example 1

In this example, we encapsulated doxorubicin, a chemotherapy drug into silk plates through solid mixing and thermoplastic molding as described in methods. We found that the polycaprolactone coating significantly decreased drug release kinetics by creating a hydrophobic water barrier.

Materials: doxorubicin (Caymen Chemical), polycaprolactone (Millipore Sigma, MW 80 k), chloroform (Millipore Sigma), silk amorphous nanoparticles made from lyophilization and analytical milling.

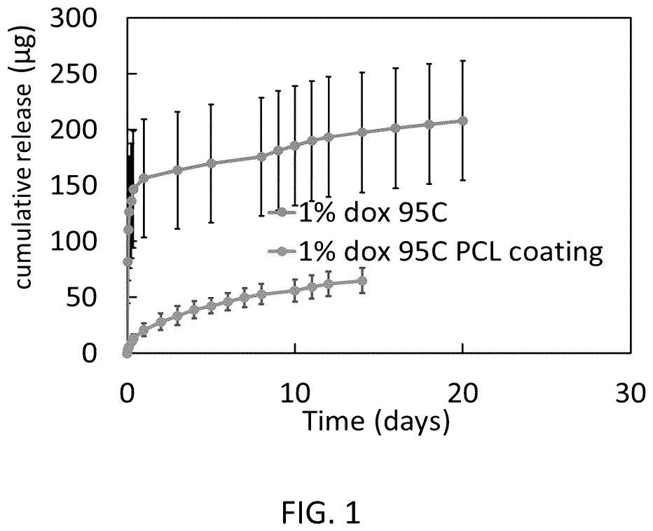

Methods: doxorubicin (1 wt %) was mixed with silk amorphous nanoparticles using analytical milling (20,000 r.p.m., 2 min, Col-Parmer). The mixed powder (50 mg) was thermoplastic molded into silk plates with doxorubicin encapsulated. Polycaprolactone was dissolved in chloroform at 5 wt %. Thermoplastic molded silk bars were dipcoated with the polycaprolactone chloroform solution (Dip Coater, Ossila Limited). Both silk plates with and without coating was immersed in 1 mL PBS solution, 37° C. The doxorubicin release kinetics were monitored by measuring the absorbance of doxorubicin at different time points. The results are shown in FIG. 1.

### Example 2

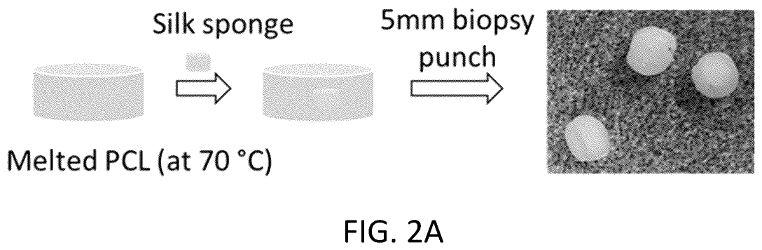

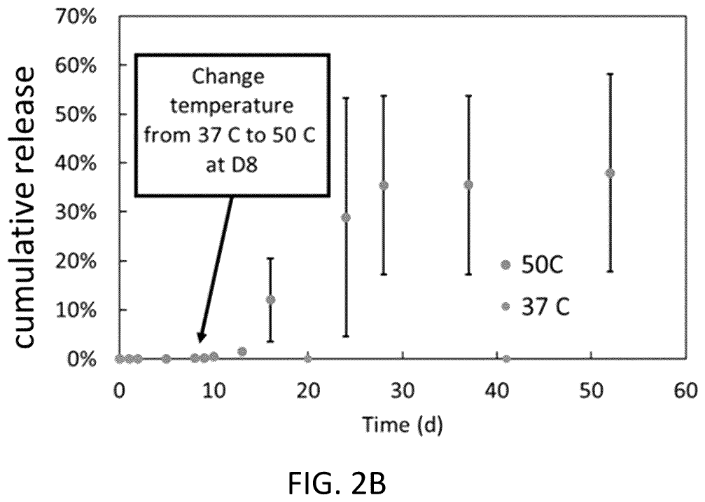

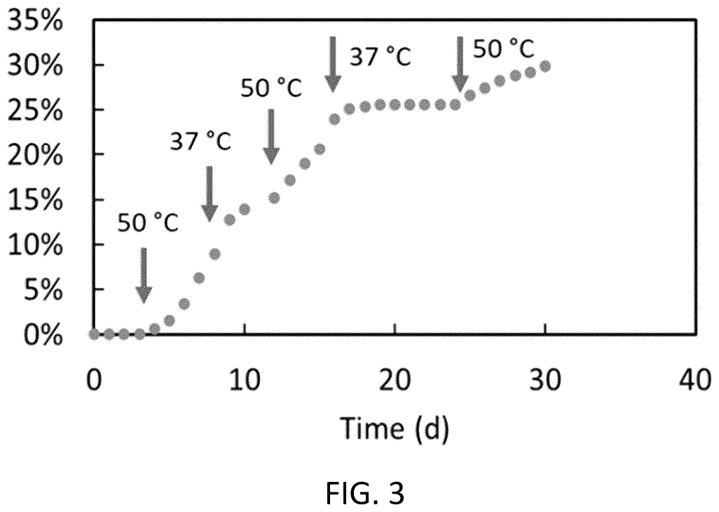

In this example, we demonstrate that polycaprolactone pellet coating on silk sponges stopped the drug release from silk sponge, by acting as a water barrier. It is also shown that the polycaprolactone pellet was temperature responsive. With temperature increased to 50° C., drug release resumed (FIG. 2). FIG. 3 shows that drug release can be stopped and resumed dependent on temperature cycles. This is because polycaprolactone has a melting temperature of ˜60° C. When the pellets are heated to 50° C., polycaprolactone polymers are in between melting and glassy state, allowing water to diffuse into the pellet, leading to the release of the small molecules. When the pellets are cooled down to 37° C., polycaprolactone becomes rigid pellet, reforming the water barrier to stop the molecules from releasing.

Materials: ceftriaxone (Caymen Chemical), silk sponge, polycaprolactone (Millipore Sigma, MW 80 k)

Methods: ceftriaxone contained silk sponges were prepared by lyophilizing silk aqueous solution mixed with ceftriaxone (1 g per 150 L silk solution (6.4 wt %)) followed by water annealing.

The PCL pellets were pre-melted in an oven (70° C.). Ceftriaxone containing silk sponges were embedded in the pre-melted PCL pellets, and biopsy punch (5 mm) was used to cut the melted PCL with silk sponges inside to form 5 mm pellet. Figure A demonstrates the process of making silk sponges embedded in the PCL pellets. The pellets were immersed in PBS solution (1 mL) at 37° C. for drug release studies. The supernatants were collected at each time point and HPLC was used to quantify the amount of ceftriaxone release at each time point. The pellets were transferred between 37° C. and 50° C. environmental chamber to control the drug release.

Equivalents and Scope. The recitation of a listing of elements in any definition of a variable herein includes definitions of that variable as any single element or combinations (or sub-combinations) of listed elements. The recitation of an embodiment herein includes that embodiment as any single embodiment or in combination with any other embodiments or portions thereof. Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1.-5. (canceled)

6. A coated silk fibroin article having controllable active agent release rates, the coated silk fibroin article comprising:
a solid silk fibroin dosage form core comprising an active agent; and
a polycaprolactone coating encapsulating the solid silk fibroin dosage form core,
wherein a core-only comparison dosage form comprising the solid silk fibroin dosage form core comprising the active agent and lacking any coating has a core-only active agent release rate,
wherein the coated silk fibroin article has a first active agent release rate when the polycaprolactone coating is above a first temperature threshold and below a second temperature threshold, wherein the second temperature threshold is a melting temperature of the polycaprolactone coating, wherein the first temperature threshold is a transition temperature below which the polycaprolactone coating is substantially water impermeable and above which the polycaprolactone coating is at least partly water permeable,
wherein the coated silk fibroin article has a second active agent release rate when the polycaprolactone coating is below the first temperature threshold, wherein the second active agent release rate is less than the first active agent release rate, and
wherein the coated silk fibroin article loses at least part of the polycaprolactone coating when the polycaprolactone coating is above the second temperature threshold and adopts a third active agent release rate between the first active agent release rate and the core-only active agent release rate, wherein the third active agent release rate increases as more of the polycaprolactone coating is lost from the coated silk fibroin article.

7. The coated article of claim 67, wherein the solid dosage form core is a silk fibroin foam.

8. The coated article of claim 67, wherein the active agent has a molecular weight of 10 kg/mol or less, 5 kg/mol or less, 1 kg/mol or less, or 750 g/mol or less.

9. The coated article of claim 67, wherein the polycaprolactone coating includes a polycaprolactone having a number average molecular weight of at least 5 kDa, at least 10 kDa, at least 20 kDa, at least 40 kDa, at least 50 kDa, at least 60 kDa, or at least 70 kDa and at most 500 kDa, at most 250 kDa, or at most 150 kDa, wherein the number average molecular weight is optionally at least 20 kDa, wherein the number average molecular weight is optionally at least 40 kDa, wherein the number average molecular weight is optionally at least 50 kDa.

10. The coated article of claim 67, wherein the first temperature threshold is higher than normal human body temperature.

11. The coated article of claim 67, wherein the first temperature threshold is at least 20° C., at least 25° C., at least 30° C., at least 35° C., or at least 40° C.

12. The coated article of claim 67, wherein the second temperature threshold is a melting point of pure polycaprolactone.

13.-21. (canceled)

22. The coated article of claim 67, the polycaprolactone coating having a substantially uniform thickness.

23. The coated article of claim 67, the polycaprolactone coating having an average thickness of between 1 m and 5 mm.

24. The coated article of claim 67, wherein the polycaprolactone coating accounts for no more than 50% by weight of the coated article and at least 1% by weight of the coated article.

25. The coated article of claim 67, the solid dosage form core and/or the polycaprolactone coating comprising an acid-activated protease.

26. The coated article of claim 25, wherein the acid-activated protease is selected from the group consisting of pepsin, a cathepsin, renin, and combinations thereof.

27. The coated article of claim 25, wherein the acid-activated protease is triggered by a local pH reduction initiated by dissolution or degradation of polycaprolactone coating.

28. The coated article of claim 67, wherein the coated article comprises a flexural strength of at least 50 MPa.

29. The coated article of claim 67, the coated article and/or the solid dosage form core having a density of at least 1.20 g/cm³.

30.-62. (canceled)

63. A method of delivering an active agent to a target location with a controlled release rate, the method comprising:
a) administering a polycaprolactone-coated dosage form to a target location, the polycaprolactone-coated dosage form having a solid dosage form core comprising an active agent and a polycaprolactone coating encapsulating the solid dosage form core; and
b) subsequent to step a), elevating temperature of the polycaprolactone-coated dosage form to above a first temperature threshold and below a second temperature threshold, wherein the second temperature threshold is a melting temperature of the polycaprolactone coating, wherein the first temperature threshold is a transition temperature below which the polycaprolactone coating is substantially water impermeable and above which the polycaprolactone coating is at least partly water permeable, thereby allowing water to pass between the target location and the solid dosage form core and providing a first active agent release rate of the active agent from the polycaprolactone-coated dosage form to the target location.

64. The method of claim 63, the method further comprising:
c) subsequent to step b), reducing the temperature of the polycaprolactone-coated dosage form at the target location to below the first temperature threshold, thereby returning the polycaprolactone coating to being substantially water impermeable and preventing water from passing between the target location and the solid dosage form core and providing a second active agent release rate of the active agent from the polycaprolactone-coated dosage form to the target location.

65. The method of claim 63, the method further comprising:
d) subsequent to step b), elevating the temperature of the polycaprolactone-coated dosage form at the target location to above the second temperature threshold, thereby at least partly melting the polycaprolactone coating and providing a third active agent release rate of the active agent from the polycaprolactone-coated dosage form to the target location.

66. The method of claim 63, wherein the polycaprolactone or other hydrophobic biocompatible polymer-coated silk fibroin article is prevented from exceeding the second temperature threshold prior to step b).

67. A coated article having controllable active agent release rates, the coated article comprising:
a solid dosage form core comprising an active agent; and
a polycaprolactone coating encapsulating the solid dosage form core,
wherein a core-only comparison dosage form comprising the solid dosage form core comprising the active agent and lacking any coating has a core-only active agent release rate,
wherein the coated article has a first active agent release rate when the polycaprolactone coating is above a first temperature threshold and below a second temperature threshold, wherein the second temperature threshold is a melting temperature of the polycaprolactone coating, wherein the first temperature threshold is a transition temperature below which the polycaprolactone coating is substantially water impermeable and above which the polycaprolactone coating is at least partly water permeable,
wherein the coated article has a second active agent release rate when the polycaprolactone coating is below the first temperature threshold, and
wherein the coated article loses at least part of the polycaprolactone coating when the polycaprolactone coating is above the second temperature threshold and adopts a third active agent release rate between the first active agent release rate and the core-only active agent release rate, wherein the third active agent release rate increases as more of the polycaprolactone coating is lost from the coated article.

68. (canceled)
